(19) United States
(12) Patent Application Publication
(10) Pub. No.: US 2006/0058825 A1
(43) Pub. Date: Mar. 16, 2006

(54) Ultrasonic surgical apparatus

(75) Inventors: Gen Ogura, Tokyo (JP); Kazuo Isono, Tokyo (JP)

Correspondence Address: Koda & Androlia, Suite 1140, 2029 Century Park East, Los Angeles, CA, 90067-2983, US

(73) Assignee: Aloka Co., Ltd.

(21) Appl. No.: 11/008,773

(22) Filed: Dec. 9, 2004

(30) Foreign Application Priority Data
Sep. 10, 2004 (JP) 2004-263258

(51) Int. Cl. A61B 17/32

(52) U.S. Cl. 606/169

(57) ABSTRACT

An ultrasonic surgical apparatus which can be used in laparoscopic surgery. The ultrasonic surgical apparatus includes an inserting unit and an operation unit. The inserting unit is inserted into a guide member called a trocar. The inserting unit includes an elongated member, a tip portion, and also a joint section provided between the elongated member and the tip portion. The tip portion includes a transducer unit and an oscillation member, and preferably further includes a clamp member and an open/close mechanism. The joint section enables the tip portion to be slanted with respect to the elongated member. The operation unit includes a mechanism for operating the joint section, and preferably further includes a mechanism for operating the clamp member. The direction of the oscillation member can be varied by the joint section. Because ultrasonic oscillation is generated in the tip portion, effective transmission of the ultrasonic oscillation to the oscillation member can be achieved.

### BACKGROUND OF THE INVENTION

1. Field of the Invention

The present invention relates to an ultrasonic surgical apparatus, and more particularly to an ultrasonic surgical apparatus for use in laparoscopic surgery.

2. Description of Related Art

In the field of surgical treatment as commonly practiced today, surgical operations using a laparoscope are common. In laparoscopic surgery, a tubular guide member called a trocar is introduced into the peritoneal cavity, and various surgical instruments are inserted through this guide member. Usually, a plurality of guide members are used simultaneously, with various instruments such as an endoscope and forceps, and an inserting unit of an ultrasonic surgical apparatus, as desired, are inserted into each guide member.

Conventional ultrasonic surgical apparatuses used for laparoscopic surgery as described above generally include an operation unit having an ultrasonic transducer provided therein and an elongated inserting unit having an oscillation transmission member or a waveguide to which ultrasonic oscillation or ultrasonic vibration is transmitted from the ultrasonic transducer. The inserting unit has, at its leading end, an oscillation member serving as an ultrasonic scalpel, an ultrasonic blade, or an ultrasonic end-effector, to which ultrasonic oscillation generated within the operation unit is transmitted. The oscillation member is brought into contact with body tissue so as to effect ultrasonic surgery including cutting and coagulating the tissue. In conventional ultrasonic surgical apparatuses as described above, ultrasonic oscillation is generated outside a human body and transmitted to the oscillation member introduced into the human body as described above. When positioning the oscillation member within the body, the inserting direction and depth of the oscillation member is adjusted by moving and locating the operation unit.

Published Japanese translation of unexamined PCT application (Kohyo) No. 8-505801 (corresponding to International Laid-Open Publication No. WO94/16631) discloses an ultrasonic surgical apparatus which, in use, sandwiches target issue between an oscillation member and a clamp member. Again, the ultrasonic transducer of the disclosed apparatus is located outside a human body.

Published Japanese translation of unexamined PCT application (Kohyo) No. 2003-527155 (corresponding to International Laid-Open Publication No. WO01/24714) also discloses an ultrasonic surgical apparatus which has an oscillation member and a clamp member. FIG. 23 and other figures of this publication show an oscillation member having a gently curving shape. Yet again, the ultrasonic transducer of the disclosed apparatus is located outside a human body.

Japanese Patent Laid-Open Publication No. Hei 11-56867 discloses an ultrasonic surgical apparatus in which a disposable ultrasonic transducer is mounted on the leading edge of a holding rod which is to be inserted in a trocar placed within a human body. The ultrasonic transducer is provided with an ultrasonic surgical blade. The leading edge of the holding rod is inserted into the ultrasonic transducer so that both are secured to each other, and the ultrasonic transducer is removable from the holding rod. The diameter of the ultrasonic transducer is greater than that of the holding rod. In other words, the ultrasonic transducer has an increased diameter. It is not possible to change the direction of the blade with respect to the holding rod when the ultrasonic transducer is coupled to the holding rod.

Japanese Patent Laid-Open Publication No. Hei 1-232948 discloses an ultrasonic surgical apparatus having an ultrasonic transducer provided at the base end (outside the body) of an inserting unit and an operation rod capable of forward/backward movement and extending through the ultrasonic transducer.

Japanese Patent Laid-Open Publication No. Hei 1-232949 also discloses an ultrasonic surgical apparatus having an ultrasonic transducer provided at the base end (outside the body) of an inserting unit and a rod-shape member (auxiliary member for cutting tissue) capable of forward and backward movement and extending through the ultrasonic transducer.

For ultrasonic surgery using a laparoscope, if the inserting unit to be inserted into the guide member is a simple rod-like member whose shape cannot be changed, the following problems occur when guiding an oscillation member serving as a surgical instrument toward a target site of the surgery. One problem, for example, is that it is difficult to bring the oscillation member into contact with tissue located at the back of certain tissue. As another example, the ultrasonic surgical operation must be performed with the inserting unit being held at a forced angle, which lowers operability of the apparatus or makes it difficult to increase safety. Even with an oscillation member having a curving shape, the above problems remain unsolved as long as the degree of the curvature is fixed. The degree of curvature must be determined such that the curving oscillation member can be inserted into the guide member. Further, when the oscillation member is greatly curved, unnecessary lateral oscillation is likely to occur.

U.S. Pat. No. 6,063,098 describes an ultrasonic surgical device having a surgical blade, a clamp member, and a joint section. The joint section enables the surgical blade and the clamp member to change their position and direction within the body. Ultrasonic oscillation generated in the ultrasonic transducer located outside the body is transmitted to the surgical blade through a long waveguide, which is formed by a plurality of members connected together so as to have flexibility by itself. The above-described structure suffers from a problem in that energy is lost during transmission of ultrasonic oscillation.

U.S. Pat. No. 5,413,107 discloses an ultrasonic probe having a joint section. However, this is not an ultrasonic surgical apparatus.

### SUMMARY OF THE INVENTION

The present invention advantageously provides an ultrasonic surgical apparatus capable of achieving increased operability and a high level of safety during ultrasonic surgery.

The present invention advantageously provides an ultrasonic surgical apparatus which advantageously achieves a high degree of freedom for positioning of a surgical unit for effecting coagulation and cutting of tissue.

The present invention advantageously provides an ultrasonic surgical apparatus in which the size of a tip portion to be inserted into a living body can be reduced.

(1) In accordance with one aspect of the present invention, there is provided an ultrasonic surgical apparatus comprising an inserting unit, and an operation unit which is provided at an end of the inserting unit to be located outside of a living body, the inserting unit including an elongated member, a tip portion provided at the leading end of the elongated member, and a joint section which varies the direction of the tip portion with respect to the elongated member, and the tip portion further including a transducer unit for generating ultrasonic oscillation, and an oscillation member to which the ultrasonic oscillation generated in the transducer unit is transmitted and which comes into contact with tissue of a living body.

With the above-described structure, the joint section provided in the inserting unit enables the tip portion to change its direction with respect to the elongated member. More specifically, the tip portion is provided with the oscillation member for effecting coagulation and cutting of body tissue, and the position and orientation of the oscillation member can be varied easily by operation of the joint section. This facilitates ultrasonic surgery toward target tissue located on the back side of certain tissue and also allows a variation of methods in which the oscillation member approaches the target tissue, thereby increasing safety. When inserting the inserting unit into a guide member and extracting the inserting unit from. the guide member, a state in which a virtual central axis in elongated member is matched to a virtual central axis in the tip portion (non-bending state of the joint section) is generally established. The joint section is formed by one or more joint mechanisms (bending mechanisms). The joint section may include a rotating mechanism for causing the tip portion to rotate about the central axis of the elongated member or may include a mechanism for causing the tip portion to oscillate in an arbitrary three-dimensional direction.

With this structure, a transducer unit is provided within the tip portion which is to be introduced into a living body, and ultrasonic oscillation is generated within the living body. As a result, unlike in the conventional devices, it is not necessary to transmit ultrasonic oscillation from the operation unit side (namely from outside the body) to the tip portion (namely to inside the body). Further, because the transducer unit is provided beyond the joint section toward the forefront end of the apparatus, the necessity of transmitting ultrasonic oscillation via the joint section can be eliminated. Consequently, a problem of energy loss caused when transmitting ultrasonic oscillation over a long distance or via the joint section to the tip portion can be solved.

Preferably, the joint section causes the tip portion to move in at least one direction with respect to the elongated member. Alternatively, the joint section causes the tip portion to move in an arbitrary direction with respect to the elongated member. The direction of movement of the tip portion is set fixedly or arbitrarily. The range in which the movement of the tip portion is allowed is set fixedly or arbitrarily.

Preferably, the tip portion further includes a clamp member for sandwiching the tissue with the oscillation member, and an open/close mechanism for causing the clamp member to open and close. With this structure, the oscillation member and the clamp member form a surgical unit. With body tissue being held by the surgical unit, ultrasonic oscillation is transmitted from the oscillation member to the body tissue for effecting coagulation and cutting of the tissue.

The tip portion may also include, in addition to the open/close mechanism, a mechanism for generating an open/close drive force. However, in order to reduce the size of the tip portion, it is desirable to provide a mechanism for generating an open/close drive force on the operation unit side and transmit the open/close drive force from the mechanism to the open/close mechanism via the joint section. Further, while a mechanism for generating a bending drive force may be provided on the joint section, in order to reduce the size of the inserting unit, it is desirable to provide such a mechanism for generating a bending drive force on the operation unit, which then transmits the bending drive force to the joint section.

A clamp member having a plurality of different clamp surfaces may be provided in the tip portion. In this case, a clamp surface to be actually used is selected by rotation of the clamp member itself, for example. Similarly, the oscillation member may have a plurality of different tissue contact surfaces. In this case, a tissue contact surface to be actually used is selected by, for example, rotation of the oscillation member itself, or by rotation of the clamp member about the oscillation member which serves as a rotation center. Also, the tip portion may include a mechanism for rotating the oscillation member and the clamp member together. While it is desirable to form each of the oscillation member and the clamp member in a linear shape, they may be gently curved.

Preferably, the operation unit includes a first driving mechanism which generates a first drive force for causing the open/close mechanism to operate, and the inserting unit further includes a first transmission mechanism for transmitting the first drive force from the first driving mechanism to the open/close mechanism. The first driving mechanism generates a drive force by user operation, or electrically.

Preferably, the first transmission mechanism includes a first transmission member extending from the elongated member to the open/close mechanism in the tip portion via the joint section, and the open/close mechanism causes the clamp member to be opened or closed in accordance with forward or backward movement of the first transmission member. It is desirable to employ, as the first transmission member, a rod member which can transmit a drive force in both directions by forward and backward movement. However, other members including a wire member, a belt member, and the like may also be used.

Preferably, the first transmission member extends along a central axis of the joint section within the joint section. It is desirable to provide the first transmission member so as to extend along the central axis of the joint section, because it is generally recognized that the route along the central axis in the joint section does not change its length or changes its length only slightly even when the joint section is subjected to bending. In other words, it is desirable that the first transmission mechanism be configured such that it is not affected by the bending movement of the joint section.

Preferably, the transducer unit has a central hole formed along the central axis thereof, and a leading end portion of the first transmission member is inserted into the central hole. With this structure, in which the first transmission member can be guided to the open/close mechanism through the interior of the transducer unit, it is possible to effectively use the dead space to thereby prevent enlargement of the size of the tip portion.

Preferably, the transducer unit further includes a lateral hole communicating with the central hole, the open/close mechanism includes a link member extending through the lateral hole, the link member including a first end portion engaged with the leading end of the first transmission member and a second end portion coupled with the clamp member, forward or backward movement of the first transmission member is transformed into rotation movement of the link member, and the rotation movement of the link member causes the clamp member to be opened and closed. With this structure, when the first transmission member moves linearly within the central hole, the link member engaged with the leading end of the first transmission member rotates and this rotation causes the clamp member to be closed or opened. The lateral hole communicates with the central hole and serves as an opening which the link member can pass through. Alternatively, a further lateral hole may be formed on the opposite side of the lateral hole with the central hole interposed between them. Such a symmetrical structure can suppress disturbance of ultrasonic oscillation and also can be advantageous in terms of manufacturing and assembling.

Preferably, the position where the link member engages with the leading end portion of the first transmission member is set to a position of a node of ultrasonic oscillation or in the vicinity thereof. With this structure, even when the above-described structure is incorporated in the transducer unit, influence of the structure on the ultrasonic oscillation can be reduced. In particular, it is possible to prevent disturbance of oscillation mode caused in accordance with the open/close state of the clamp member.

Preferably, the central hole is a non through hole having a closed leading end. While it is possible to form the whole oscillation member as a hollow member, it is desirable to form the whole or the main portion of the oscillation member as a solid member.

Preferably, the operation unit includes a second driving mechanism which generates a second drive force for causing the joint section to operate, and the inserting unit further includes a second transmission mechanism for transmitting the second drive force from the second driving mechanism to the joint section. The second driving mechanism is formed as a mechanism which generates the second drive force by user operation or electrically.

Preferably, the second transmission mechanism includes a second transmission member extending from the operation unit to the joint section, and movement of the second transmission member causes the joint section to operate. While it is desirable to use, as the second transmission member, a pair of wire members as will be described below, other members may also be used.

Preferably, the second transmission member includes a pair of wire members, and the pair of wire members is formed by two wire members passing through two positions, respectively, which, in the joint section, are displaced from each other in the bending direction of the wire members, with the central axis of the joint section being disposed therebetween. Alternatively, it is preferable that the second transmission member includes a first pair of wire members and a second pair of wire members, the first pair of wire members including two wire members disposed at two positions, respectively, which, in the joint section are displaced from each other in a first bending direction of the wire members, with the central axis of the joint section being disposed therebetween, and the second pair of wire members including two wire members disposed at two positions, respectively, which, in the joint section, are displaced from each other in a second bending direction of the wire members, with the central axis of the joint section being disposed therebetween.

With the above structure, when one wire member is pulled in, the route length of the other wire member is extended to cause the joint section to bend in the direction toward the one wire member. When the other wire member is pulled in, the apparatus operates vice versa. When the joint section can be bent in a plurality of directions, it is desirable to provide a pair of wire members for each bending direction.

Preferably, the oscillation member has a hook shape at the leading end. With this hook shape, it is possible to perform ultrasonic surgery while body tissue is being hooked on the leading end of the oscillation member. The leading end of the oscillation member may have a shape other than the hook shape. Preferably, the cross section of the tip portion is of substantially the same size as the cross section of the elongated member. It is desirable that the tip portion does not have an increased size with respect to the elongated member in order to allow smooth insertion and smooth extraction of the inserting unit into and out of the guide member. Specifically, it is desirable that the cross section of the tip portion (in particular, the drive unit excluding the surgical unit) has the same size as the cross section of the elongated member (or that the former is smaller than the latter). The tip portion may have a tapered shape as a whole.

(2) In accordance with another aspect of the present invention, there is provided an ultrasonic surgical apparatus comprising an inserting unit to be inserted into a tubular guide member, and an operation unit which is provided at an end of the inserting unit to be located outside of a living body, the inserting unit including an elongated member, a tip portion provided at the leading end of the elongated member, and a joint section which varies the direction of the tip portion with respect to the elongated member, and the tip portion further including a transducer unit for generating ultrasonic oscillation, an oscillation member to which the ultrasonic oscillation generated in the transducer unit is transmitted and which comes into contact with tissue of a living body, a clamp member for sandwiching the tissue with the oscillation member, and an open/close mechanism for causing the clamp member to open and close.

With the above structure, the body tissue is sandwiched by a surgical unit formed by the oscillation member and the clamp member, and ultrasonic oscillation is transmitted from the oscillation member to the tissue in this state. Thus, coagulation and/or cutting of the tissue is performed. Because the direction of the surgical unit can be varied by the joint section, the operability and safety of the apparatus can be increased. Further, because ultrasonic oscillation is generated beyond the joint section toward the forefront end of the apparatus, the necessity for transmitting ultrasonic oscillation via the joint section can be eliminated. Thus, because ultrasonic oscillation which is generated can be transmitted to the oscillation member directly or via a short route, high level transmission efficiency can be achieved.

Preferably, the operation section includes a first driving mechanism having a first operation member which is operated by a user, for generating a first drive force which causes the open/close mechanism to operate, and a second driving mechanism having a second operation member which is operated by a user, for generating a second drive force which causes the joint section to operate, and the inserting unit further includes a first transmission mechanism for transmitting the first drive force from the first driving mechanism to the open/close mechanism, and a second transmission mechanism for transmitting the second drive force from the second driving mechanism to the joint section. With this structure, a user can easily control the open/close operation of the clamp member and the bending operation of the joint section.

Preferably, the first transmission mechanism is provided in the joint section and the transducer unit along central axes of the joint section and the transducer unit. The length of the route on the central axis does not change or changes only slightly even when the joint section is subjected to bending. It is therefore desirable to provide the first transmission mechanism on the central axis.

Preferably, the first transmission mechanism includes a rod member, the second transmission mechanism includes a wire member, the inserting unit further includes a plurality of signal lines connected with the transducer unit, and the joint section includes a first guide structure for guiding the rod member of the first transmission mechanism, a second guide structure for guiding the wire member of the second transmission mechanism, and a third guide structure for guiding the plurality of signal lines extracted from the transducer unit. It is desirable that each guide structure smoothly guides the bending movement of each member extending through the joint section when the joint section is subjected to bending movement. It is also desirable that each guide structure smoothly guides forward/backward movement of each member extending through the joint section. The joint section may be covered with a bendable sheath tube or the like as necessary, so that tissue can be prevented from being caught in the joint section, for example.

Preferably, each of the signal lines includes an amount of slack for allowing movement of the joint section. Such a slack can solve the problem that the movement of the joint section is limited by the plurality of signal lines.

While it is preferable that the above structures are adopted in a surgical apparatus for use in ultrasonic surgery only, they may also be used in a surgical apparatus for use in both ultrasonic surgery and electrosurgery. For the latter, a high frequency signal for electrosurgery may be supplied from the operation unit side to the oscillation member via the joint section.

As described above, according to the above structures, the operability of the apparatus can be improved and safety of the ultrasonic surgery can be ensured. In addition, the degree of freedom for the position and posture of the surgical unit which performs coagulating, cutting, and the like of the tissue can be increased. Also, it is possible to reduce the size of the tip portion to be inserted into a body.

### BRIEF DESCRIPTION OF THE DRAWINGS

Preferred embodiments of the present invention will be described in further detail with reference to the following drawings, wherein:

FIG. 1 is a conceptual view showing an ultrasonic surgical apparatus according to a first embodiment of the present invention;

FIG. 2 is an X-Z cross sectional view of the inserting unit of the first embodiment of the present invention;

FIG. 3 is an Y-Z cross sectional view of the inserting unit of the first embodiment of the present invention;

FIG. 4 is an X-Z cross sectional view of the operation unit of the first embodiment of the present invention;

FIG. 5 is an Y-Z cross sectional view of the operation unit of the first embodiment of the present invention;

FIG. 6 is a conceptual view showing an ultrasonic surgical apparatus according to a second embodiment of the present invention;

FIG. 7 is a view showing the inserting unit of the second embodiment of the present invention in a linear state;

FIG. 8 is a view showing the inserting unit of the second embodiment of the present invention in a bending state;

FIG. 9 is an Y-Z cross sectional view of the inserting unit of the second embodiment of the present invention;

FIG. 10 is a cross sectional view taken along line A-A′ of FIG. 9;

FIG. 11 is an Y-Z cross sectional view of the operation unit of the second embodiment of the present invention;

FIG. 12 is a conceptual view showing an ultrasonic surgical apparatus according to a third embodiment of the present invention;

FIG. 13 is an Y-Z cross sectional view of the inserting unit of the third embodiment of the present invention; and

FIG. 14 is a cross sectional view taken along line B-B′ of FIG. 13.

### DETAILED DESCRIPTION OF PREFERRED EMBODIMENTS

Preferred embodiments of the present invention will be described in detail with reference to the drawings.

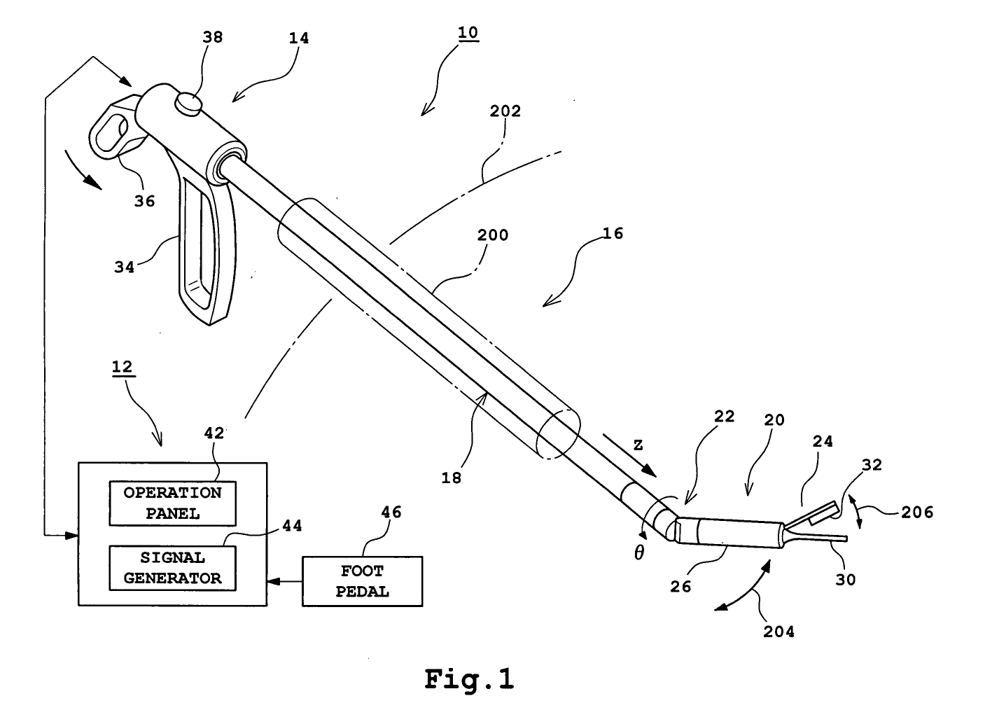

FIGS. 1 to 5 show a first embodiment of an ultrasonic surgical apparatus according to the present invention. An ultrasonic surgical apparatus performs coagulation and cutting with respect to tissue of a living body.

Referring to FIG. 1, an ultrasonic surgical apparatus is formed roughly by a handpiece 10 and a control unit 12 which are electrically connected to each other via a cable or the like. The ultrasonic surgical apparatus is used for a surgical operation employing a laparoscope. In FIG. 1, a tubular guide member (trocar) 200 is inserted, through a surface of a living body (an abdomen surface) 202, into a peritoneal cavity.

The handpiece 10 includes an operation unit 14 to be located outside the body and an inserting unit to be inserted into the body. In laparoscopic surgery, the inserting unit 16 is used while it is being inserted into the guide member 200, as shown in FIG. 1. The ultrasonic surgical apparatus shown in FIG. 1 can also be used in normal open surgery for which a guide member is not used. The inserting unit 16 includes an elongated member 18 serving as a main shaft portion and a tip portion 20 provided at the leading end of the inserting unit 16. The inserting unit 16 also includes a joint section 22 provided spanning over the elongated member 16 and the tip portion 20 (or between the elongated member 16 and the tip portion 20). The elongated member 18 is of a cylindrical shape extending in the Z direction, and the tip portion 20 has a similar elongated shape. A surgical unit 24 is mounted toward the leading end of the tip portion. The outer diameter of the elongated member 18 is the same as the outer diameter of a drive unit 26 of the tip portion 20. In other words, the tip portion 20 does not have an increased diameter in the embodiment.

The tip portion 20 includes the surgical unit 24 and the drive unit 26, as described above. The drive unit 26 includes a transducer unit and an open/close mechanism, as will be detailed below. The surgical unit 24 includes an oscillation member (an ultrasonic end-effector) 30 and a clamp member 32. The clamp member 32 is opened and closed under control of the open/close mechanism. An ultrasonic surgical operation of tissue is performed with the tissue being sandwiched between the oscillation member 30 and the clamp member 32. In FIG. 1, the open/close movement of the clamp member 32 is shown by arrows 206.

The joint section 22 is a mechanism for allowing slanting or bending movement of the tip portion 20 with respect to the elongated member 18. The joint section 22 is formed by one or more joint mechanisms, each of which is capable of bending by itself. FIG. 1 shows a typical example of a joint section 22 having a single joint mechanism, which allows the tip portion 20 to slant in the predetermined direction, as shown in arrows 204 in FIG. 1. In the structure example shown in FIG. 1, assuming that the direction of the central axis of the elongated member 18 is defined as the Z direction, it is possible to cause the tip portion 20 to bend on a plane defined by the Z direction and the predetermined direction which is orthogonal to the Z direction. The movable range of the tip portion 20 is determined as ±60°, for example, although a greater or smaller range be selected as preferable.

The θ direction is defined as the direction around the Z direction axis. A separate mechanism for rotating the joint section 22 (or the tip portion 20) in the θ direction may be provided. This structure enables the tip portion 20 to bend at an arbitrary angle and in an arbitrary direction. Further, a ball joint mechanism may be provided as the joint section 22. More specifically, the apparatus may be configured so as to allow slant movement of the tip portion 20 in an arbitrary three-dimensional direction.

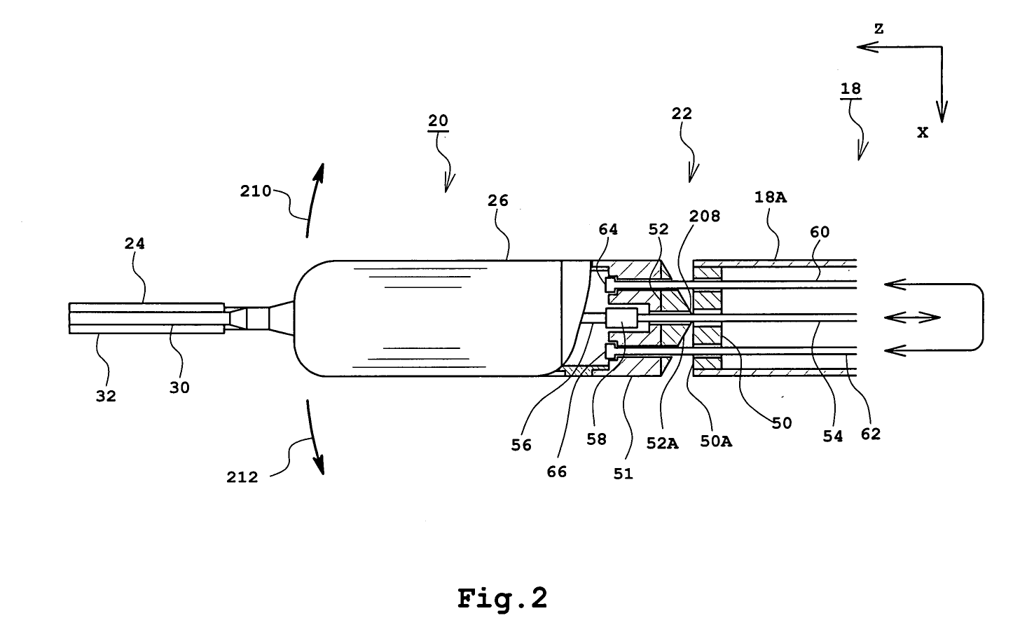

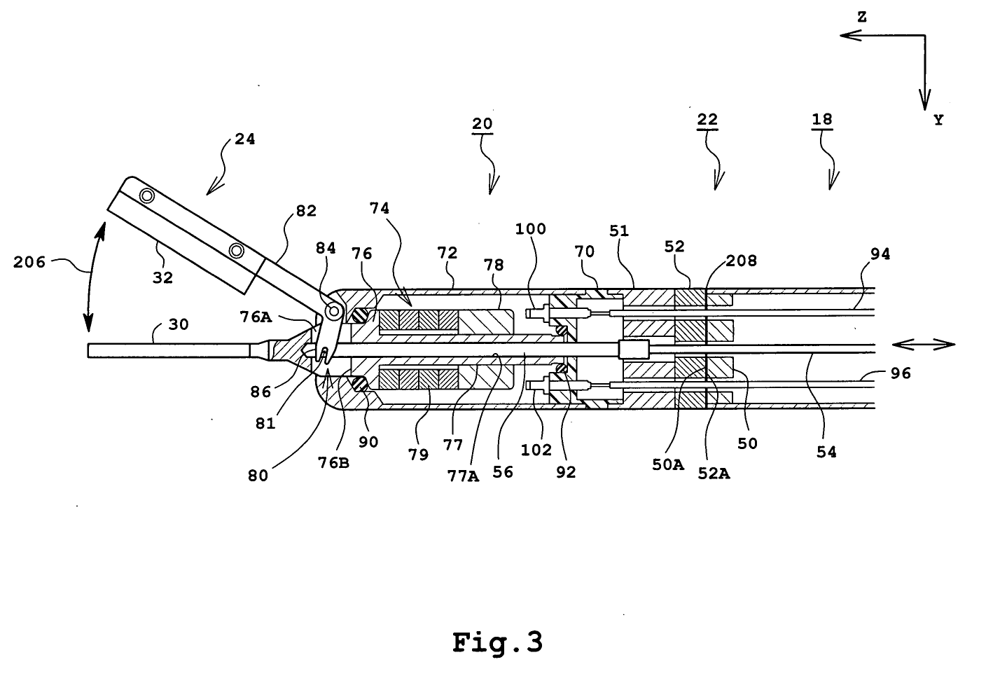

The joint section 22 is conceptually shown in FIG. 1. In addition to the mechanisms shown in FIGS. 2 and 3 described below, the joint section 22 may also adopt various known mechanisms having, for example, a structure similar to a human backbone. It is desirable to cover the entire joint section 22 with a sheath tube, that is a flexible tube in order to prevent catching of a tissue by the joint section 22 and entrance of a body fluid into the joint section 22. Alternatively, the joint section 22 may be covered with a deformable member having a bellows structure. The tip portion 20 may be detachably mounted with respect to the elongated member 18.

In a device with the above structure, the clamp member 32 may be configured such that a plurality of clamp surfaces having different shapes are provided and a clamp surface to be actually used is selected by rotating the clamp member 32. Similarly, the oscillation member 30 may be configured such that a plurality of tissue contact surfaces having different shapes are provided and a tissue contact surface to be actually used is selected by rotating the oscillation member 30 or by rotating the clamp member 32 about the oscillation member 30.

The operation unit 14 is located outside the human body during ultrasonic surgery, as shown, and is held and operated by the user. The operation unit 14 includes a grip 34 and a lever member 36. The user may insert the fingers of one hand, other than the thumb, through the opening of the grip 34 and insert their thumb through the opening of the lever member 36, for example. When the user shuts their hand in this state, the lever member 36 is moved toward the grip 34. The drive force generated at this time is transmitted as a drive force for closing the clamp member 32. This will be described in detail below with reference to FIGS. 2 to 5.

The operation unit 14 includes an operation knob 38. By rotating the operation knob 38 clockwise or counterclockwise, the joint section 22 is driven to thereby cause the tip portion 20 to bend in one direction or in the opposite direction, as will be described in detail with reference to FIGS. 2 and 3. A variety of structures may be employed for the operation unit 14, among which FIG. 1 shows only one example. It is possible to adopt a mechanism for opening or closing the clamp member 32 by operating a lever similar to the trigger of a pistol. While the drive force for the joint section 22 and the drive force for the clamp member 32 are both generated by user operation in this embodiment, these drive forces may be generated electrically using a drive motor or the like.

In order to perform ultrasonic surgery, the inserting unit 16 is inserted into the guide member 200. Prior to this insertion, it is usually necessary to make the center axis of the elongated member 18 coincide with the center axis of the tip portion 20, namely to place the tip portion 20 in a non-slanted state. In addition, prior to the insertion of the inserting unit 16, the clamp member 32 is placed in a closed state. With the above preparation, the inserting unit 16 is ready for insertion into the guide member 200. Specifically, it is possible to prevent the tip portion 20 or the clamp member 32 from striking against the inlet of the guide member 200 at the time of insertion. By adjusting the position and posture of the inserting unit 16 while it is inserted into a body cavity or by operating the joint section 22 to adjust the slanting angle of the tip portion 20, it is possible to enable the surgical unit 24 to approach the target tissue at an appropriate angle from an appropriate position. This makes it possible to perform safe ultrasonic surgery with respect to target tissue located at the back of a certain tissue. The target tissue is sandwiched between the clamp member 32 and the oscillation member 30, and is subjected to coagulation and cutting when ultrasonic oscillation is transmitted from the oscillation member 30 to the tissue in this state. When the ultrasonic surgery is completed, the tip portion 20 is placed back to a non-slant state, namely returned to a center position, and the clamp member 20 is closed. Then, the inserting unit 16 is pulled out of the guide member 200.

In order to ensure that the tip portion 20 is not in a slanted state at the time of insertion and extraction, it is desirable to provide a marker indicative of the center position on the operation knob 38. It is also preferable to provide a micro switch on the joint section 22 for electrically detecting that the tip portion 20 is at the center position and to provide a display indicating the detection result on the operation unit 14 or the control unit 12. Similarly, it is desirable to provide a display indicating that the clamp member 32 is closed or opened on the operation unit 14 or the control unit 12.

The control unit 12 includes an operation panel 42 and a signal generator 44, as shown in FIG. 1. The operation panel 42 is formed by various input switches, a display device, or the like. The signal generator 44 is a module which, in response to an ON signal supplied from a foot pedal 46, generates an electrical drive signal for generating ultrasonic oscillation. The foot pedal 46 is operated by being stepped by the user of the handpiece 10.

While the ultrasonic surgical apparatus shown in FIG. 1 is used only for the purpose of ultrasonic surgery, the present invention is also applicable to a surgical apparatus which can be used for both ultrasonic surgery and electrosurgery. In that case, a signal line for supplying a high frequency signal for electrosurgery to the tip portion 20 can be provided in addition to the above structure. When electrosurgery is performed, it is possible to operate the oscillation member 30 in monopolar mode or operate the oscillation member 30 and the clamp member 32 in bipolar mode.

A specific example structure of the tip portion 20 and the joint section 22 will be described with reference to FIGS. 2 and 3. The X and Y directions are defined as directions orthogonal to the Z direction. The X direction and the Y direction are orthogonal to each other. FIG. 2 shows an X-Z cross section and FIG. 3 shows an Y-Z cross section.

Referring to FIG. 2, the tip portion 20 is mounted on the leading end of the elongated member 18, as described above. The tip portion 20 includes the oscillation member 30 and the clamp member 32 serving as the surgical unit 24. The joint section 22 is formed spanning over both the leading end of the elongated member 18 and the trailing end of the tip portion 20.

A case 18A of the elongated member 18 is provided, on the edge portion, with a member 50 having an end surface 50A which is a flat surface orthogonal to the Z axis. On the other hand, the tip portion 20 includes a base 51 which is provided, at its end, with an abut member 52 having an isosceles triangle cross section. As shown, the abut member 52 has two slant surfaces 52A, and the line where these two slant surfaces 52A contact forms a ridge-line 208. The abut member 52 pivots about the ridge-line 208 on the end surface 50A. Due to the pivot movement of the abut member 52, the whole tip portion 20 performs rocking movement, namely slanting movement in the directions shown in the arrows 210 and 212.

As shown in FIG. 2, first and second wire members 60 and 62 are leading from the elongated member 18 toward the tip portion 20, with end portions 64 and 66 thereof being fixed in the base 51. The first wire member 60 and the second wire member 62 pass through two positions in the elongated member 18, respectively, which are displaced from the center of the elongated member 18 in the X direction. In order to cause each wire member 60 or 62 pass through, corresponding guide holes are formed in each of the member 50, the abut member 52 and the base 51. The first wire member 60 and the second wire member 62 are connected with each other in the operation unit, so that backward movement of one member causes the other member to move forward.

Consequently, when the first wire member 60 is moved backward or retreated to the right in FIG. 2, the tip portion 20 rotates about the ridge-line 208 serving as a rotation axis in the direction shown in the arrow 210. On the other hand, when the second wire member 62 is moved backward or retreated to the right in FIG. 2, the tip portion 20 rotates about the ridge-line 208 serving as a rotation axis in the direction shown in the arrow 212. The tip portion 20 can be rotated until one of the slant surfaces 52A comes in complete contact with the end surface 50A. When it is desired to further increase the slanting angle of the tip portion 20, such a large angle can be achieved by providing and connecting a plurality of mechanisms as shown in FIG. 2 in series, or by coupling, in series, a plurality of block members having a structure like human backbone. Further, a first rod member 54 is provided along the central axis of the elongated member 18. The leading end of the first rod member 54 is coupled, through guide holes formed at the centers of the member 50, the abut member 52 and the base 51, respectively, to the trailing end of a second rod member 56 via a joint member 58.

The first rod member 54 and the second rod member 56 together form a first transmission mechanism for opening and closing the clamp member 32. The first wire member 60 and the second wire member 62 described above together form a second transmission mechanism for causing the joint section 22 to bend.

Referring to FIG. 3, the specific structure of the tip portion 20 will be described. FIG. 3 shows an Y-Z cross section of the tip portion 20 and the joint section 22. The tip portion 20 includes a case 72, and an intermediate member 70 which is formed by a resin member between the case 72 and the base 51 described above. As shown, a transducer unit 74 is provided within the case 72. The transducer unit 74 includes front block 76, a bolt 77, and a rear block 78. A plurality of stacked ring-shape piezoelectric plates 79 are sandwiched between the front block 76 and the rear block 78. In other words, the transducer unit 74 forms a so-called bolt joint type ultrasonic transducer. In the embodiment shown in FIG. 3, the front block 76 is integrally formed with the bolt 77. The threaded portion formed on the rear block 78 engages with the threaded portion of the bolt 77. In FIG. 3, a plurality of electrode plates in the transducer unit 74 are not shown.

The front block 76 is coupled with the oscillation member 30. The front block 76 and the oscillation member 30 may be integrally formed into a single member or may be formed separately. As shown in FIG. 3, the transducer unit 74 includes a central hole 77A serving as a non-through hole along the center axis thereof. The central hole 77A specifically extends to the trailing end of the oscillation member 30. At the position of the leading end of the central hole 77A, lateral holes 76A and 76B are formed in the Y direction. The lateral holes 76A and 76B open to the opposite directions and have the same shape. Although it is essentially necessary to provide only the lateral hole 76A in order to cause an arm member 82 which will be described below to move, it is possible to effectively prevent generation of unnecessary lateral oscillation by forming the two symmetrical lateral holes 76A and 76B as in the present embodiment. Here, the leading end (a closed surface) of the central hole 77A is positioned such that the forward movement of the second rod member 56, namely the opening movement of the clamp member 32 can be sufficiently secured. In the structure shown in FIG. 3, the leading end of the central hole 77A is positioned at the portion of the front block 76 having a larger diameter.

An open/close mechanism 80 will next be described. The second rod member 56 has, at its leading end portion, a pin 81, with which the arm member 82 is engaged. More specifically, the first end of the arm member 82 has a bifurcated shape 86 and the pin 81 is inserted into the recess of the bifurcated part. The arm member 82 rotates about a rotation axis 84. On the second end of the arm member 82, the clamp member 32 is provided.

When the first rod member 54 is moved backward to the right in FIG. 3, the second rod member 56 is also moved backward to the right accordingly. As a result of the engagement of the pin 81 and the bifurcated portion 86, this backward movement is transmitted to the arm 82, which then rotates counterclockwise about the rotation axis 84. In other words, the arm 82 moves toward the closing direction. When the first rod member 54 is moved forward to the left in FIG. 3, on the other hand, the second rod member 56 is also moved forward to the right. As a result of the engagement of the pin 81 and the bifurcated portion 86, the arm 82 member rotates clockwise about the rotation axis 84. In other words, the arm 82 moves toward the opening direction. By employing the first transmission mechanism formed by the two rod members connected in series and causing the first transmission mechanism to move forward or backward as described above, it is possible to control the opening and closing of the clamp member 32 easily and reliably at the base end portion of the apparatus, namely outside the human body.

As shown in FIG. 2, the first rod member 54 passes through the central axis of the joint section 22. The second rod member 56 is formed by a material which is bendable and transmits drive force in the forward and backward directions. While the first rod member 54 also bends according to the bending of the joint section 22, the first rod member 54 can be caused to smoothly move forward and backward, namely the clamp member 32 can be caused to smoothly open and close, even when the joint section 22 is in a bending state. In order to reduce the load applied to the first rod member 54 when it is bent, a flexible tube may be provided for protecting the bending portion of the first rod member 54. Such a protecting structure can be similarly provided to the first and second wire members 60 and 62 shown in FIG. 2 and also to signal lines 94 and 96 which will be described below.

In FIG. 3, the intermediate member 70 is provided with two electrode members 100 and 102 which are spaced apart from each other by a predetermined distance in the Y direction. These electrode members 100 and 102 function as positive and negative electrode members, respectively, and are electrically connected to a plurality of electrodes (not shown) in the transducer unit 74 via a plurality of signal lines. A pair of signal lines 94 and 96 are inserted through the elongated member 18, as shown. The leading end of the signal line 94 is electrically connected to the electrode member 100, and the leading end of the signal line 96 is electrically connected to the electrode member 102. The signal lines 94 and 96 pass through the through holes formed in the member 50, the abut member 52, and the base 51, respectively. Each of the signal lines 94 and 96 includes a predetermined amount of slack, so that the movement of the joint section 22 cannot be hindered even when a tensioning force or the like is applied to the signal lines 94 and 96 due to the bending of the joint section 22. The signal lines 94 and 96 may be connected to the electrode members 100 and 102 using soldering, or using a connector.

The transducer unit 74 is held within the case 72 by means of O-rings 90 and 92. The distance between the leading surface of the oscillation member 30 and the rear surface of the bolt 77 corresponds to a half the wavelength of ultrasound. At the position of a node of the oscillation or in the vicinity thereof, the holding position for the O-ring 90 and the lateral holes 76A and 76B are located. This structure can minimize the effect of the O-ring 90 onto the ultrasonic oscillation and also the effect caused by formation of the lateral holes 76A and 76B. It is desirable that the O-ring 92 holds the rear end of the bolt 77 loosely. In addition to the function of holding the transducer unit 74 as described above, the O-ring 90 also has a sealing function. This is also true to the O-ring 92.

The open/close mechanism 80 of FIG. 3 is shown for the illustrative purpose only and other structures may be used. For example, the arm member 82 may be driven using a wire or the like. With the structure shown in FIG. 3, in which the second rod member 56 is inserted within the central hole 77A provided along the central axis of the bolt 77 and in which the first end portion of the arm member 82 is coupled with the rod member within the interior space of the transducer unit 74, it is possible to effectively utilize the dead space so that the size of the tip portion 20 can be reduced.

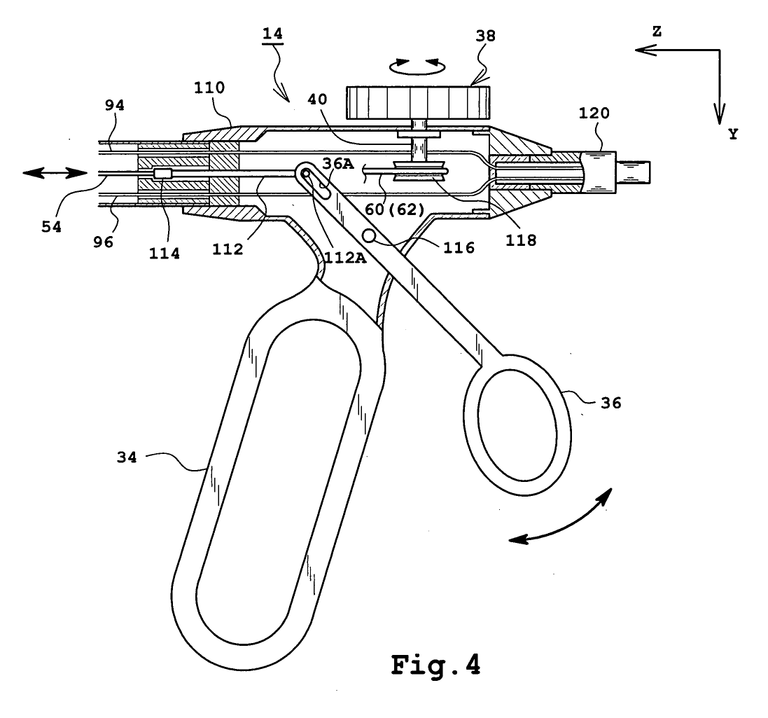

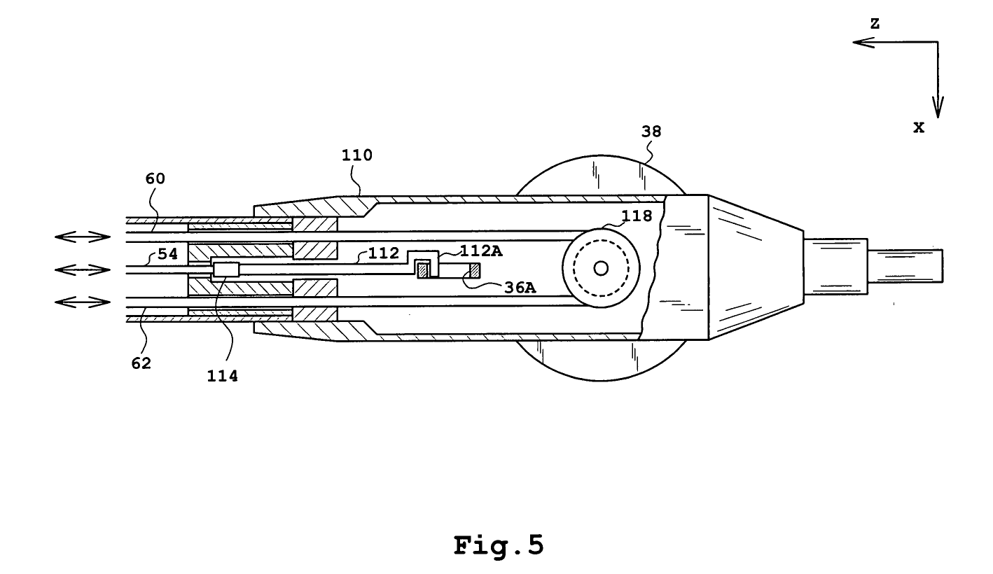

A specific example of the operation unit 14 shown in FIG. 1 will be described with reference to FIGS. 4 and 5. FIG. 4 shows an Y-Z cross section of the operation unit 14 and corresponds to FIG. 3 with regard to the direction of the cross section. FIG. 5 shows an X-Z cross section of the operation unit 14 and corresponds to FIG. 4 with regard to the direction of the cross section.

The operation unit 14 includes the grip 34 and the lever member 36, as described above. Within the case 110, the lever member 36 has an elongated hole 36A formed at its operative end. A hook 112 A, which forms one end of a link member 112, engages the elongated hole 36A. The other end of the link member 112 is coupled to the first rod member 54 via a joint member 114. Accordingly, when the lever member 36 rotates, the first rod member 54 moves forward or backward depending on the direction of rotation. In FIG. 4, numeral 116 denotes the rotation axis of the lever member 36.

The signal lines 94 and 96 are guided from the elongated member through the interior of the operation unit 14 to a connector portion 120. A cable used for electrically connecting the handpiece with the control unit is coupled to the connector portion 120.

A pulley 118 is coupled to an axis 40 of the operation knob 38. The pulley 118 has a wire member wounded around, the wire member forming the first wire member 60 and the second wire member 62 described above. Therefore, with this structure, rotation of the operation knob 38 causes the wire members to advance or retreat in accordance with the rotation direction of the knob 38, whereby a drive force is transmitted to the joint section.

As described above, the operation unit 14 includes a mechanism for generating a drive force for the clamp member and a drive force for the joint section. More specifically, the drive force for the clamp member is generated by movement of the lever member 36, and the drive force for the joint section is generated by the rotation of the operation knob 38. These drive forces may instead be generated using a drive motor or the like, as described above.

FIG. 5 shows an X-Z cross section of the operation unit 14. The hook 112A engages with the elongated hole 36A, as described above, and in FIG. 5, the movement of the hook 112A to the right and left has been transformed to the movement of the link member 112 to the right and left. When the joint section is formed by a plurality of joint mechanisms so as to realize a plurality of bending directions, a plurality of operation knobs may be provided corresponding to the respective bending directions. This structure is advantageous in that the tip portion can be bent in any three-dimensional direction. Further, when a rotating mechanism for rotating the joint section itself is provided at the leading end of the elongated member, an operation knob for actuating the rotating mechanism may be provided on the operation unit 14.

When the present invention is configured as in the above-described embodiment, because the transducer unit is disposed beyond the joint section toward the leading end of the apparatus, it is not necessary to transmit the ultrasonic oscillation generated outside a living body to the tip portion, especially through the joint section. It is also possible to operate the joint section and the open/close mechanism in the operation unit. In this case, the open/close mechanism can be advantageously operated regardless of the operation of the joint section. Further, the structure in which the rod member is inserted through the central hole formed along the central axis of the transducer unit can achieve an advantage of reduction in size of the tip portion.

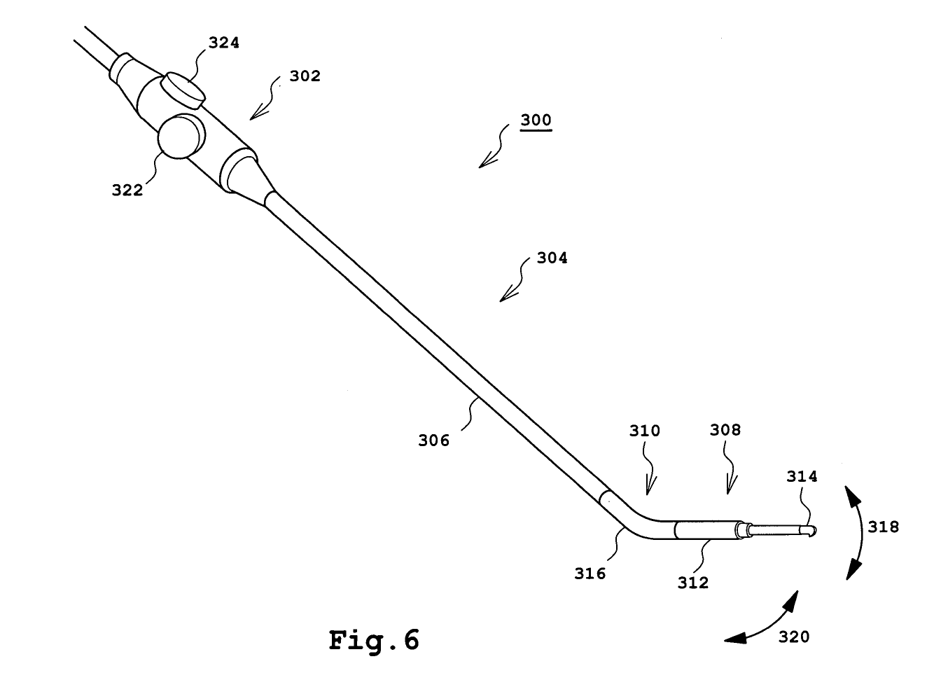

A second embodiment of the present invention will next be described with reference to FIGS. 6 to 11.

FIG. 6 is a perspective view of an ultrasonic surgical apparatus according to the second embodiment. In FIG. 6, only a handpiece is shown and a control unit is omitted. As in the first embodiment, a handpiece 300 includes an operation unit 302 and an inserting unit 304. When the apparatus is in use, the operation unit 302 is located outside a body, and the inserting unit 302 is inserted into a guide member (not shown). Namely, the inserting unit 304 is introduced into the body. The inserting unit 304 includes an elongated member 306, a tip portion 308, and a joint section 310 provided between the elongated member 310 and the tip portion 308. The joint section 310 allows the direction of the tip portion 308 to be changed in an arbitrary direction with respect to the elongated member 306. Assuming that the direction of insertion corresponds to the Z direction, it is possible to cause the joint section 310 to bend both in the X direction (see numeral 320) and in the Y direction (see numeral 318) orthogonal to the Z direction. Further, the tip portion 308 may be bent in other directions.

The tip portion 308 includes a drive unit 312 and an oscillation member 314. The drive unit 312 includes a transducer unit which will be described below. The oscillation member 314 comes in contact with tissue of a living body and transmits ultrasonic oscillation to the tissue for effecting coagulation and cutting of the tissue. The oscillation member 314 has a hook at its leading end, which catches tissue of a living body for effecting ultrasonic surgery in that state. In the second embodiment, a clamp member is not provided on the tip portion 308. The oscillation member 314 may have any of various shapes at its leading end.

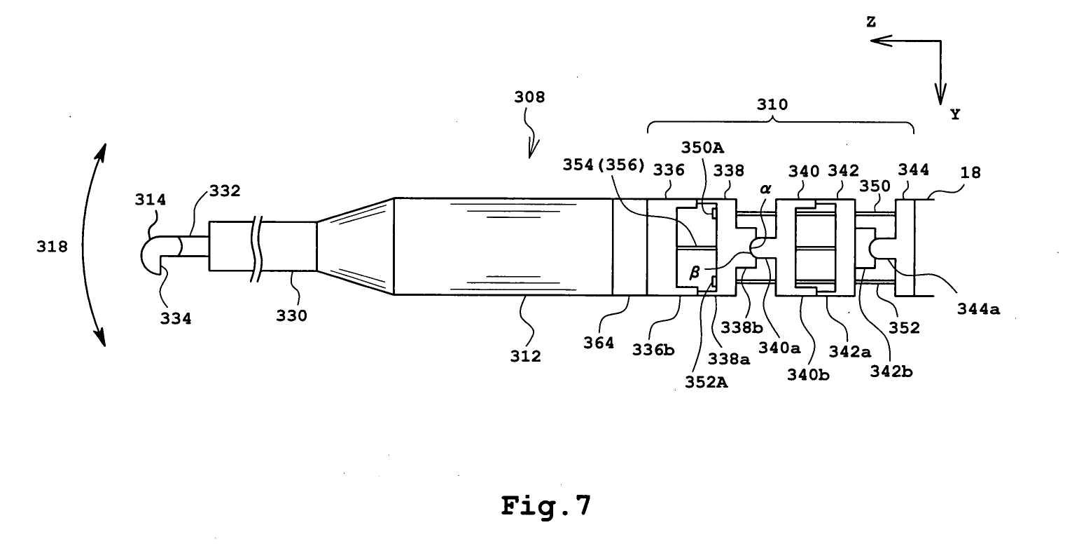

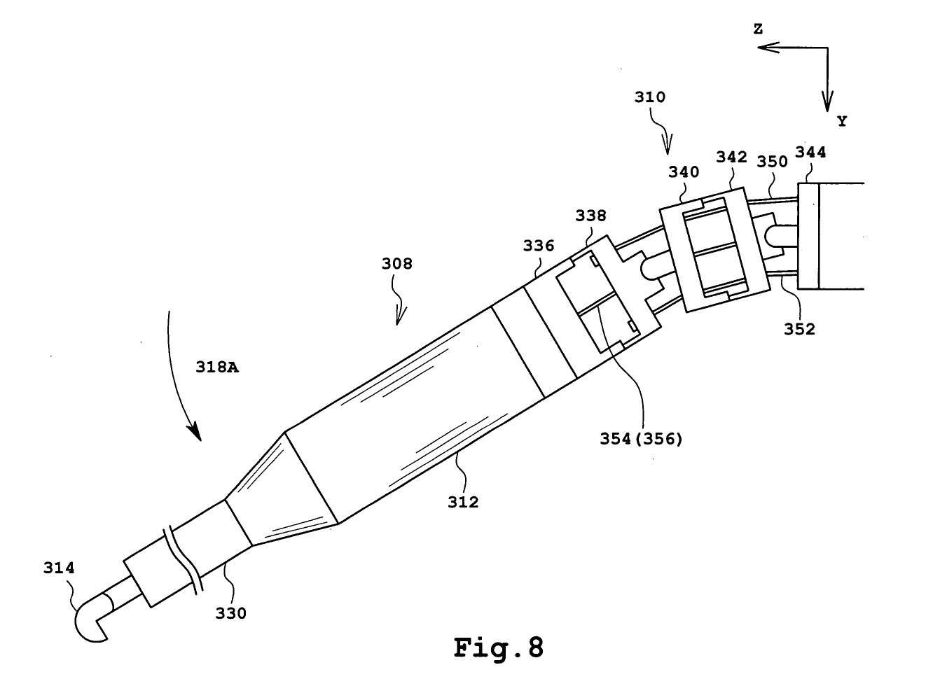

FIG. 7 shows an enlarged view of the tip portion 308 and the joint section 310. In FIG. 7, as well as in FIGS. 8 to 10, a plurality of signal lines connected to the transducer unit within the tip portion are omitted. Further, in FIG. 7, as well as in FIGS. 8 to 10, a flexible outer tube covering the whole joint section is also omitted.

In the tip portion 308 shown in FIG. 7, a cover 330 is provided at the leading end of the drive unit 312. The oscillation member 314 is formed by a shaft portion 332 and a hook portion 334, with most part of the shaft portion 332 being covered with the cover 330. In the example shown in FIG. 7, the joint section 310 includes a plurality of blocks 336, 338, 340, 342, and 344 which are coupled in series. The forefront block 336 is fixed to the tip portion 308. More specifically, the block 336 functions as a base formed at the trailing end of the tip potion 308, in other words, the block 336 forms a part of the tip portion 308. The last block 344 is fixed to the elongated member 306. More specifically, the block 344 functions as a cap provided at the front end of the elongated member 306, namely forms a part of the elongated member 306.

The block 336 includes two contact portions 336b spaced apart from each other in the Y direction by a predetermined distance. Each contact portion 336b is formed to project to the right in FIG. 7. The block 338 includes two contact portions 338a spaced apart by a predetermined distance from each other in the Y direction. Each contact portion 338a is formed to project to the left in FIG. 7. Each of the two contact portions 336b has a curved concave portion and each of the two contact portions 338a has a curved convex portion. Between the two blocks 336 and 338, two Y-axis rotation mechanisms which are spaced apart by a predetermined distance from each other in the Y direction are formed. Each of the Y-axis rotation mechanisms is formed by a concave portion and a convex portion which are contact with each other in such a manner that they can mutually rotate about the Y axis.

The block 338 includes two contact portions 338b spaced apart from each other in the X direction by a predetermined distance. Each contact portion 338b is formed to project to the right in FIG. 7. The block 340 includes two contact portions 340a spaced apart from each other in the X direction by a predetermined distance. Each contact portion 340a is formed to project to the left in FIG. 7. Each of the two contact portions 338b has a curved concave portion (α) and each of the two contact portions 340a has a curved convex portion (β). Between the two blocks 338 and 340, two X-axis rotation mechanisms which are spaced apart from each other in the X direction by a predetermined distance are formed. Each of the X-axis rotation mechanisms is formed by a concave portion (α) and a convex portion (β) which are coupled with each other in such a manner that they can mutually rotate about the X axis.

The structure which is similar to that provided between the blocks 336 and 338 as described above is also provided between the block 340 and the block 342. Specifically, the block 340 includes two contact portions 340b which are spaced apart from each other in the Y direction by a predetermined distance, and the block 342 includes two contact portions 342a which are spaced apart from each other in the Y direction by a predetermined distance. Further, the structure which is similar to that provided between the blocks 338 and 340 as described above is also provided between the block 342 and the block 344. Specifically, the block 342 includes two contact portions 342b which are spaced apart from each other in the X direction by a predetermined distance, and the block 344 includes two contact portions 344a which are spaced apart from each other in the X direction by a predetermined distance.

Two wire members 350 and 352 are provided through the plurality of blocks 338, 340, 342, and 344 in such a manner that the two wire members 350 and 352 are spaced apart from each other by a predetermined distance in the Y direction and are disposed in parallel to each other. The leading ends 350A and 352A of the two wire members 350 and 352 are fixed to the second block 338. Further, two wire members 354 and 356 are provided through the plurality of blocks 338, 340, 342, and 344 in such a manner that the two wire members 354 and 356 are spaced apart from each other by a predetermined distance in the X direction and are disposed in parallel to each other. The leading ends (not shown) of the two wire members 354 and 356 are fixed to the intermediate member which will be described below or to the forefront block 336.

As can be understood from the structure described above, when one of the two wire members 350 and 352 is pulled to the right in FIG. 7, the joint section 310 is bent in the up-down direction as shown by arrows 318, which causes the tip portion 308 to change its direction on the Y-Z plane. FIG. 8 shows the resultant state caused by pulling the wire member 352. When one of the wire members 354 and 356 is pulled to the right in FIG. 7, on the other hand, the joint section 310 is bent in the direction passing through the sheet plane in FIG. 7, which causes the tip portion 308 to change its direction on the X-Z plane. Thus, by pulling one of the two wire members 350 and 352 and one of the two wire members 354 and 356 simultaneously to the right in FIG. 7, the joint section 310 can be bent in any three-dimensional direction, thereby allowing the tip portion 308 to be directed in arbitrary three-dimensional direction.

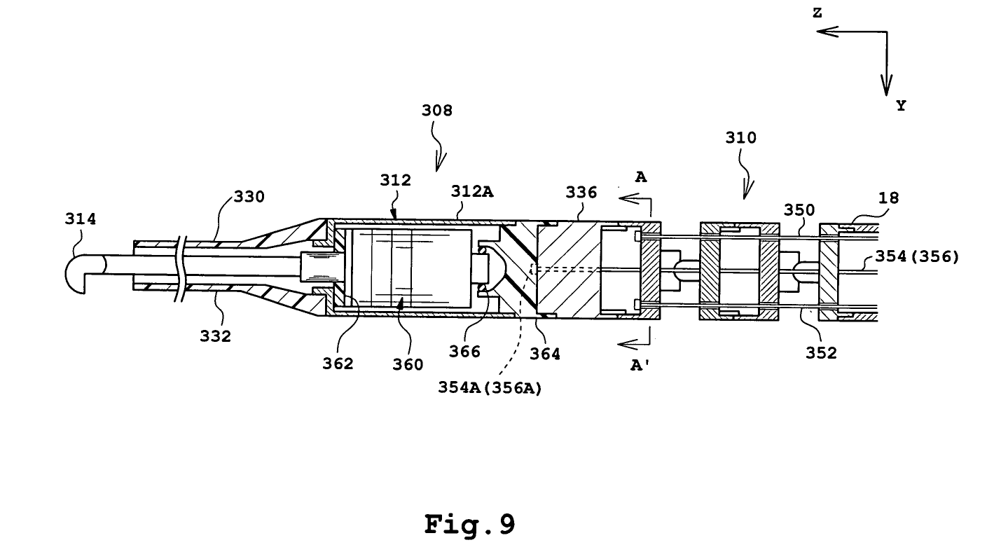

FIG. 9 is a cross sectional view of the tip portion 308 and the joint section 310 described above. A transducer unit 360, which has a bolt joint type ultrasonic transducer, is provided within a case 312 of the tip portion 308. The rear portion of the transducer unit 360 is held by an O-ring 366 provided on the intermediate member 364. The front of the transducer unit 360 is held by the case 312 via a ring-shape packing 362. The oscillation member 314 is fixed to the leading end of the transducer unit 360, so that ultrasonic oscillation generated in the transducer unit 360 is transmitted directly to the oscillation member 314. The oscillation member 314 includes a shaft portion 332 covered with a cover 330, the components having a gap between them. The leading ends 354A and 356A of the two wire members 354 and 356 for use in X-direction driving are fixed to the intermediate member 364 (or the forefront block 336).

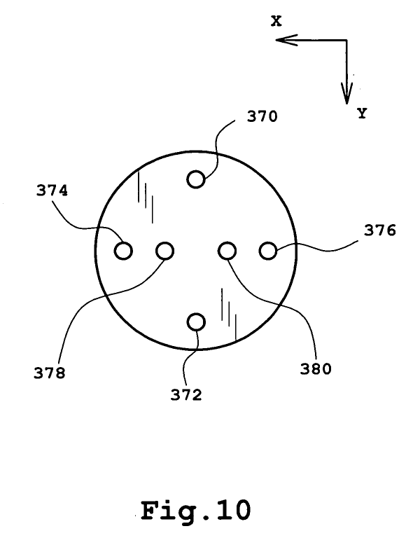

FIG. 10 is a cross sectional view showing an A-A′ cross section (a cross section of the second block) in FIG. 9. In FIG. 10, numerals 370 and 372 indicate two through holes through which the two wire members 350 and 352 shown in FIG. 9 pass, respectively, numerals 374 and 376 indicate two through holes through which the two wire members 354 and 356 shown in FIG. 9 pass, respectively, and numerals 378 and 380 indicate two through holes through which two signal lines pass, respectively.

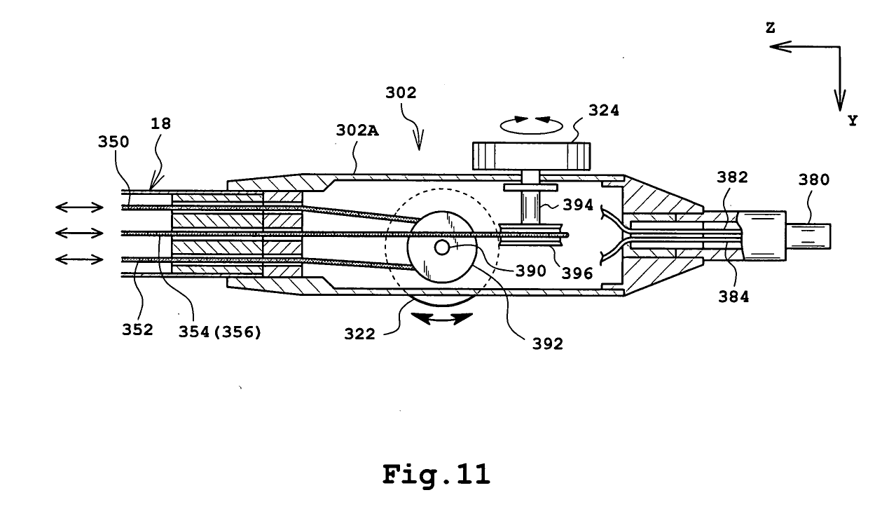

FIG. 11 is a cross sectional view of the operation unit 302 in the second embodiment described above. A pulley 392 is coupled with a shaft 390 of an operation knob 322. The wire members 350 and 352 are formed into a single wire member within the operation unit 302, and the single wire member is wound around the pulley 396. Similarly, a pulley 396 is coupled with a shaft 394 of an operation knob 324. The wire members 354 and 356 are formed into a single wire member within the operation unit 302, and the single wire member is wound around the pulley 376. Accordingly, rotation of the operation knob 322 causes the two wire members 350 and 352 to move in the opposite directions, thereby making the joint section bend in the Y direction. Further, rotation of the operation knob 324 causes the two wire members 354 and 356 to move in opposite directions, thereby making the joint section bend in the X direction. By rotating both of the two operation knobs 322 and 324, it is possible to tilt the joint section in any three-dimensional direction. A change in the bending direction of the joint section depends on the ratio of rotation amounts of the two operation knobs 322 and 324. It is desirable to provide indicators showing the rotation amount on each of the operation knobs 322 and 324. The two signal lines 382 and 384 are connected to the connector 380.

As in the first embodiment, according to the second embodiment, the direction of the tip portion can be changed by the joint section within a body, and because ultrasonic oscillation can be generated in the tip portion, the necessity to transmit ultrasonic oscillation via the joint section can be eliminated, thereby allowing effective transmission of ultrasonic oscillation to living tissue. In particular, because the tip portion can be directed in any three-dimensional direction, the operability and integrity of the apparatus can be enhanced.

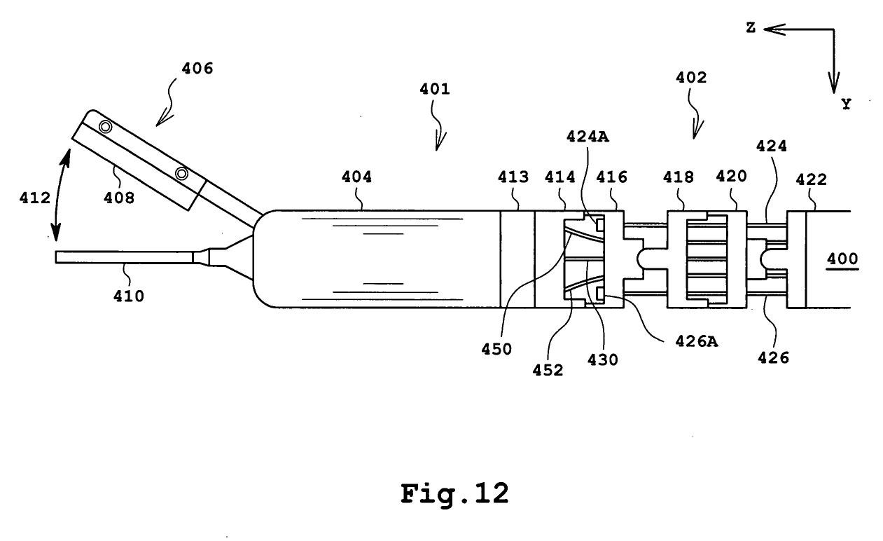

A third embodiment of the present invention will next be described with reference to FIGS. 12 to 14.

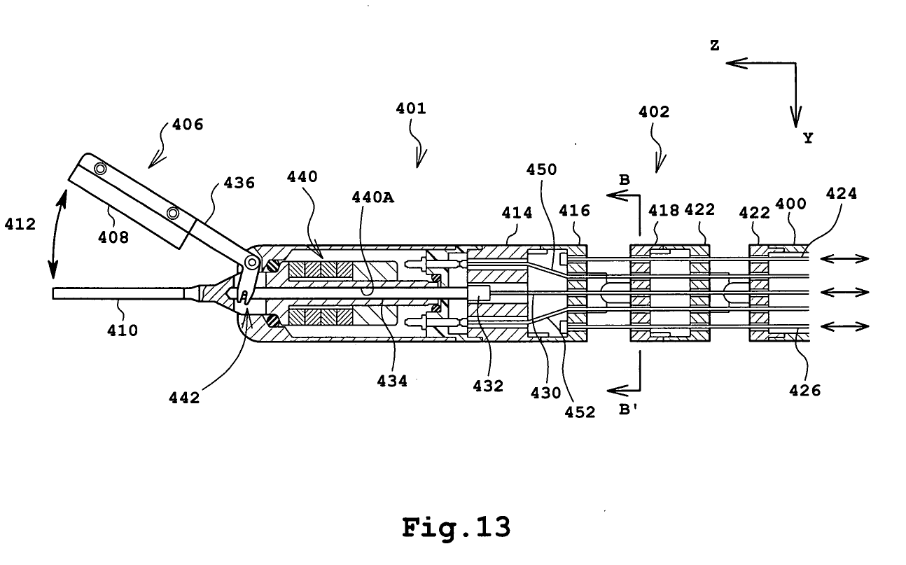

FIG. 12 shows a tip portion 401 and a joint section 402 of an ultrasonic surgical apparatus. In FIG. 12, and also in FIG. 13, a tube covering the joint section is omitted. Referring to FIG. 12, the joint section 402 is provided between the tip portion 401 and the elongated member 400. The joint section 402 has basically the same structure as the joint section in the second embodiment described above, except that the joint section 402 additionally includes a rod member 430 which will be described below. The joint section 402 is formed by a plurality of blocks 414, 416, 418, 420, and 422 which are coupled in series. Two wire members 424 and 426 for Y-direction driving pass through the blocks 416, 418, 420, and 422, and the leading ends 424A and 426A of the two wire members 424 and 426 are fixed to the second block 416. On the other hand, two wire members (not shown) for X-direction driving pass through the plurality of blocks 414, 416, 418, 420, and 422, and the leading ends (not shown) of the two wire members are fixed to an intermediate member 413 or to the forefront block 414. In addition, two signal lines 450 and 452 pass through the plurality of blocks 414, 416, 418, 420, and 422. The tip portion 401 has a structure similar to that of the tip portion in the first embodiment. Specifically, the tip portion 401 includes a drive unit 404 and a surgical unit 406. The surgical unit 406 includes an oscillation member 410 and a clamp member 408.

FIG. 13 is a cross sectional view of the tip portion 401 and the joint section 402. A transducer unit 440 has a central hole 440A formed along the Z direction. A second rod member 434 is provided in the central hole 440A in such a manner that the second rod member can move forward and backward. The second rod member 434 has a pin at its leading end, and a bifurcated edge of an arm member 436 engages with the pin. When the first rod member 430 is moved forward or backward, the second rod member 434 also moves accordingly. This forward or backward movement is transformed into rotating movement of the arm member 436, which results in open/close movement of the clamp member 408 (see arrows 412).

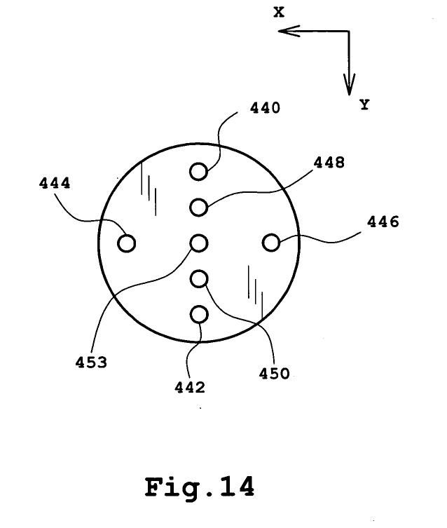

FIG. 14 shows a B-B′ cross section (a cross section of the second block) in FIG. 13. In FIG. 14, numerals 440 and 442 indicate two through holes through which two wire members 424 and 426 for Y-direction driving shown in FIG. 13 pass, respectively, numerals 444 and 446 indicate two through holes through which two wire members for X-direction driving pass, respectively, and numerals 448 and 450 indicate two through holes through which two signal lines 450 and 452 shown in FIG. 13 pass, respectively. Through a through hole 453 formed at the center, the first rod member 430 shown in FIG. 13 passes.

In the third embodiment, it is desirable to provide an operation unit (not shown) which is similar to the operation unit 14 shown in FIG. 1. More specifically, the operation unit includes a mechanism for controlling the open/close movement of the clamp member, a mechanism for causing the joint section to bend in the Y direction, and a mechanism for causing the joint section to bend in the X direction.

According to the third embodiment, as in the first and second embodiments, the direction of the tip portion can be changed by the joint section within a body, and because ultrasonic oscillation can be generated in the tip portion, the necessity to transmit ultrasonic oscillation via the joint section can be eliminated, thereby allowing effective transmission of ultrasonic oscillation to living tissue. In particular, because the tip portion can be directed in any three-dimensional direction, the operability and integrity of the apparatus can be enhanced. Also, the tip portion can be reduced in size by utilizing the dead space within the transducer unit.

While the preferred embodiments of the present invention have been described using specific terms, such description is for illustrative purposes only, and it is to be understood that changes and variations may be made without departing from the spirit or scope of the appended claims.

What is claimed is:

1. An ultrasonic surgical apparatus comprising:
an inserting unit; and
an operation unit which is provided at an end of the inserting unit to be located outside of a living body,
the inserting unit including an elongated member, a tip portion provided at the leading end of the elongated member, and a joint section which varies the direction of the tip portion with respect to the elongated member, and
the tip portion further including a transducer unit for generating ultrasonic oscillation, and an oscillation member to which the ultrasonic oscillation generated in the transducer unit is transmitted and which comes into contact with tissue of a living body.

2. An ultrasonic surgical apparatus according to claim 1, wherein
the joint section causes the tip portion to move in at least one direction with respect to the elongated member.

3. An ultrasonic surgical apparatus according to claim 1, wherein
the joint section causes the tip portion to move in an arbitrary direction with respect to the elongated member.

4. An ultrasonic surgical apparatus according to claim 1, wherein
the tip portion further includes:
a clamp member for sandwiching the tissue with the oscillation member; and
an open/close mechanism for causing the clamp member to open and close.

5. An ultrasonic surgical apparatus according to claim 4, wherein
the operation unit includes a first driving mechanism which generates a first drive force for causing the open/close mechanism to operate, and
the inserting unit further includes a first transmission mechanism for transmitting the first drive force from the first driving mechanism to the open/close mechanism.

6. An ultrasonic surgical apparatus according to claim 5, wherein
the first transmission mechanism includes a first transmission member extending from the elongated member to the open/close mechanism in the tip portion via the joint section, and
the open/close mechanism causes the clamp member to open or close in accordance with forward or backward movement of the first transmission member.

7. An ultrasonic surgical apparatus according to claim 6, wherein
the first transmission member extends along a central axis of the joint section within the joint section.

8. An ultrasonic surgical apparatus according to claim 6, wherein
the transducer unit has a central hole formed along the central axis thereof, and
a leading end portion of the first transmission member is inserted into the central hole.

9. An ultrasonic surgical apparatus according to claim 8, wherein
the transducer unit further includes a lateral hole communicating with the central hole,
the open/close mechanism includes a link member extending through the lateral hole, the link member including a first end portion engaged with the leading end of the first transmission member and a second end portion coupled with the clamp member,
forward or backward movement of the first transmission member is transformed into rotation movement of the link member, and
the rotation movement of the link member causes the clamp member to open and close.

10. An ultrasonic surgical apparatus according to claim 9, wherein
the position where the link member engages with the leading end portion of the first transmission member is set to a position of a node of ultrasonic oscillation or in the vicinity thereof.

11. An ultrasonic surgical apparatus according to claim 8, wherein
the central hole is a non through hole having a closed leading end.

12. An ultrasonic surgical apparatus according to claim 1, wherein
the operation unit includes a second driving mechanism which generates a second drive force for causing the joint section to operate, and
the inserting unit further includes a second transmission mechanism for transmitting the second drive force from the second driving mechanism to the joint section.

13. An ultrasonic surgical apparatus according to claim 12, wherein
the second transmission mechanism includes a second transmission member extending from the operation unit to the joint section, and
movement of the second transmission member causes the joint section to operate.

14. An ultrasonic surgical apparatus according to claim 13, wherein
the second transmission member includes a pair of wire members, and
the pair of wire members is formed by two wire members passing through two respective positions, which, in the joint section, are displaced from each other in the bending direction of the wire members, with the central axis of the joint section being disposed therebetween.

15. An ultrasonic surgical apparatus according to claim 13, wherein
the second transmission member includes a first pair of wire members and a second pair of wire member,
the first pair of wire members includes two wire members disposed at two respective positions, which, in the joint section, are displaced from each other in a first bending direction of the wire members, with the central axis of the joint section being disposed therebetween, and
the second pair of wire members includes two wire members disposed at two respective positions, which, in the joint section, are displaced from each other in a second bending direction of the wire members, with the central axis of the joint section being disposed therebetween.

16. An ultrasonic surgical apparatus according to claim 1, wherein
the oscillation member has a hook shape at the leading end.

17. An ultrasonic surgical apparatus according to claim 1, wherein
the cross section of the tip portion is of substantially the same size as the cross section of the elongated member.

18. An ultrasonic surgical apparatus comprising:
an inserting unit to be inserted into a tubular guide member; and
an operation unit which is provided at an end of the inserting unit to be located outside of a living body,
the inserting unit including an elongated member, a tip portion provided at the leading end of the elongated member, and a joint section which varies the direction of the tip portion with respect to the elongated member, and
the tip portion further including a transducer unit for generating ultrasonic oscillation, an oscillation member to which the ultrasonic oscillation generated in the transducer unit is transmitted and which comes into contact with tissue of a living body, a clamp member for sandwiching the tissue with the oscillation member, and an open/close mechanism for causing the clamp member to open and close.

19. An ultrasonic surgical apparatus according to claim 18, wherein
the operation section includes:
a first driving mechanism provided with a first operation member which is operated by a user, for generating a first drive force which causes the open/close mechanism to operate; and
a second driving mechanism provided with a second operation member which is operated by a user, for generating a second drive force which causes the joint section to operate, and
the inserting unit further includes:
a first transmission mechanism for transmitting the first drive force from the first driving mechanism to the open/close mechanism; and
a second transmission mechanism for transmitting the second drive force from the second driving mechanism to the joint section.

20. An ultrasonic surgical apparatus according to claim 19, wherein
the first transmission mechanism is provided in the joint section and the transducer unit along central axes of the joint section and the transducer unit.

21. An ultrasonic surgical apparatus according to claim 19, wherein
the first transmission mechanism includes a rod member,
the second transmission mechanism includes a wire member,
the inserting unit further includes a plurality of signal lines connected with the transducer unit, and
the joint section includes a first guide structure for guiding the rod member of the first transmission mechanism, a second guide structure for guiding the wire member of the second transmission mechanism, and a third guide structure for guiding the plurality of signal lines extracted from the transducer unit.

22. An ultrasonic surgical apparatus according to claim 21, wherein
each of the signal lines includes an amount of slack for allowing movement of the joint section.
